(19) United States
(12) Patent Application Publication
(10) Pub. No.: US 2026/0155231 A1
(43) Pub. Date: Jun. 4, 2026

(54) Video Diary and Analysis of Emotion-Text Mismatch

(71) Applicant: GAIA AG, Hamburg (DE)

(72) Inventors: Tristan Zindler, Hamburg (DE); Bernhard Wellhöfer, Hamburg (DE); Mario Weiss, Hamburg (DE)

(21) Appl. No.: 18/966,583

(22) Filed: Dec. 3, 2024

(51) Int. Cl. G16H 20/70; G16H 10/60; G16H 40/67

(52) U.S. Cl. CPC G16H 20/70; G16H 10/60; G16H 40/67

(57) ABSTRACT

A computerized CBT therapy system includes a mobile computing device; a messaging app; a computer with access to the messaging app; a communication channel between the mobile computing device and the computer; a message received by the computer from the mobile computing device; software for extracting audio, video and/or text data from the message, and for producing a summary; a database for storing a history of messages and summaries; software with access to the database for generating an assessment of the message or of a conversation including the message; a database of curated replies, which may be processed to generate a numerical representation of each reply; software for numerically representing the assessment and/or the message and for matching the assessment and/or the message to one or more curated replies; and software for generating a reply to the message; and software on the computer for skeptically analyzing the reply.

### TECHNICAL FIELD

The invention relates to software and applications thereof that is configured to provide cognitive behavioral therapy, and more particularly, to improvements in the cognitive behavioral therapy systems which allow them to be used to provide professional services.

### BACKGROUND

Presently, generative artificial intelligence systems (“GenAI”) are prevalent. Such systems use statistical guessing to produce a most likely correct response to a prompt. They lack rigor in the algorithms that generate their responses, they are prone to mistaken guesses called “hallucinations”, and there is not a high likelihood that a particular response will be “correct” in a useful sense of that term. This problem is further compounded where the inputs to the system are deliberately or accidentally incorrect or flawed in some way. Accordingly, GenAI is not appropriate for results-sensitive tasks.

An example of a results-sensitive task is Cognitive Behavioral Therapy (CBT), sometimes referred to as “talk therapy”. In the CBT treatment modality, a patient or client converses with a trained professional to enhance the patient's functioning with any of a range of psychological disorders including depression, PTSD, etc.

Treatment sessions are traditionally held in person in a comfortable setting between therapist and patient in order to promote communication and engagement. CBT treatment may further incorporate the use of a “diary” or other written, audio, or visual record maintained by the patient covering the time periods between treatment sessions. The treatment seeks to help patients become more self-aware and recognize factors that influence their emotional well-being, and also to encourage supportive behaviors and activities so patients can reach their own emotional balance.

It is generally known that a series of regular CBT sessions is necessary to reinforce treatment initiatives until patients themselves become aware of improvements in their emotional state.

Ubiquitous Internet connectivity and the rise of mobile computing devices have made it possible for CBT to be consumed by patients without actually visiting a therapists'office, and/or for the “diary” record system to be maintained on the mobile computing device and accessible to the therapist or treatment provider. The patient's own environment and schedule can be more easily accommodated to not only leverage the patient's own comfort, but also to expand delivery of CBT services and the responsiveness of a treatment provider to the patient.

Computerized therapy systems are known, including systems for providing CBT, but these systems are only “intelligent” in the sense that they have the ability to answer a limited number of questions or provide a limited amount of information. Additionally, such systems have been only text-based. They either cannot accept inputs other than text, or they only provide replies in text, or both. So, the efficacy of existing computerized CBT systems is limited at least because the full range of a therapist's observations and experience cannot be used for treatment.

Further, current generation computerized therapy systems, including systems which may be operated as “chat bots,” have trouble with logic and reasoning because they are fundamentally statistical guessing machines that produce the most “likely” response to a prompt. For example, existing systems may have trouble counting how many “r” letters are in the word “strawberry”. The reasons for these troubles are fairly simple and also are fundamental to how these systems work (i.e., existing systems may represent “strawberry” as two or three tokens and count how many tokens have an “r”). When handling signals of intense emotional valence rather than spelling words, the potential for over-simplification by an unsupervised computerized therapy system could be counter-productive or even dangerous.

It would be desirable to provide a computerized CBT system that could interpret patient inputs and generate responses with a relatively high likelihood of being “correct” as in usable for a results-sensitive purpose. Specifically, the pattern-recognition capabilities of these systems may be leveraged to provide early identification of incongruities in what a patient may “say” about their condition relative to what a patient's condition “appears” to be. Examples of results-sensitive purposes include dialogs that provide an end-user with therapeutic or advisory results; e.g., talk therapy, legal counseling, business advisement.

### SUMMARY

According to aspects of the present disclosure, a system is disclosed in which a computerized CBT system is operated by a partner. The computerized CBT system (herein also referred to as the “system”) provided includes a mobile computing device; a messaging app running on the mobile computing device; a computer with access to the messaging app; a communication channel established between the mobile computing device and the computer; a message received by the computer from the mobile computing device over the communication channel; software executing in the computer for extracting at least one of audio, video and text data from the message, and for producing a summary; a database accessible by the computer for storing a history of the messages and summaries; software executing in the computer with access to the database (and, optionally, environmental factors) for generating an assessment of the message or of a conversation including the message; a database of curated replies, which may be processed to generate a numerical representation of each reply (optionally, the numerical representations may be stored with the replies); software executing in the computer for numerically representing the assessment (and/or the message) and for matching the assessment (and/or the message) to one or more replies within the database, based on distances between the numerical representations; software executing in the computer for generating a reply to the message using at least one of the message, the summary, the assessment, and/or any matching curated content; and software executing in the computer for skeptically analyzing the reply and either returning it to the reply generating software for revision or forwarding it to the communication channel for transmission to the mobile computing device; wherein, once the reply is forwarded to the communication channel it is added to the database of messages to update a conversation including the message and the reply.

In some embodiments, a computer transmits one or more prompts to a mobile computing device encouraging a partner (variously referred to herein as a “patient” or a “user”) to submit video recordings. Said prompts may be sent periodically. The partner may create video recordings using the mobile computing device; in particular, by using a camera in communication with the mobile computing device in further communication with an application on the mobile computing device. When the partner creates a video recording, the mobile computing device may transmit the video recording along with extracted text and/or audio to a computer. The computer may be in communication with a database for storing video messages, where those video messages may be from the instant partner or anonymized ‘other’ partners which the system has interacted with or otherwise obtained from third-party databases. The computer is capable of analyzing previous video messages submitted by the same partner or the aforementioned ‘other’ partners. The computer includes software for analyzing previous video messages and to perform a comparison of the extracted content relative to the word choices made by the partner and the partner's facial expressions and tone of voice (the “expressive element”) to determine whether the content correlates with or deviates from the expressive element of the video message and, if necessary, for generating and transmitting a computerized CBT reply to the mobile computing device.

According to other aspects of the present disclosure, a computerized CBT system is provided that includes a summarizer that is configured to receive one or more messages from a partner in at least one of audio, video, and text modalities, wherein the summarizer is further configured to produce and update a case summary based at least on the one or more messages; an inner voice that is configured to produce and update an assessment of the situation based at least on the case summary and a set of professional knowledge; and a composer that is configured to produce a reply to the partner based at least on the case summary and the assessment.

According to another aspect of the present disclosure, the computerized CBT system may include a supervisor that is configured to provide feedback to the composer regarding the reply, wherein the composer is further configured to update the reply in response to the feedback. For example, the supervisor may be configured to provide the feedback based at least on the set of professional knowledge.

According to another aspect of the present disclosure, the computerized CBT system may include a curated content injection system that is configured to receive the assessment and to provide curated content to the composer based at least on the assessment.

According to another aspect of the present disclosure, the summarizer may be further configured to produce and update the case summary based also on environmental factors.

According to another aspect of the present disclosure, the inner voice may be further configured to provide at least one motivational question to the composer based at least on the case summary, the set of professional knowledge, and the assessment.

According to another aspect of the present disclosure, the summarizer may be further configured to provide at least one gap-filling question to the composer based at least on the case summary.

According to another aspect of the present disclosure, the summarizer also may be configured to provide the at least one gap-filling question based also on the assessment.

Thus, aspects of the present disclosure can provide a computerized CBT system that is available 24/7 to provide conversation partners with continuous contact or to provide an analytical comparative system to identify incongruities in patient responses. The system can be realized through a mobile text interface, for example, by texting a given number. The system may further be configured to utilize the built-in camera and microphone of a mobile computing device. Given the capabilities of speech-to-text and text-to-speech, as well as the ability for speaking video generation from 2-D still images and text, voice and video interfaces also are contemplated.

Such a system can provide partners (e.g., patients) timely and consistent support, regardless of time or location. By using advanced agent-based systems to deliver personalized responses, the system can focus on the individualized needs of partners, enhancing the accessibility and effectiveness of support.

By way of example, an embodiment of the present disclosure may be as follows: A computer sends daily alters to a mobile computing device. A patient operating the mobile computing device views one such alert and is prompted to record a “selfie” style video of themselves in which the patient's face is visible. The video may be in the form of a “diary” where the patient discusses their mental health and/or speaks candidly regarding other personal matters. The video recorded by a patient should capture at least a portion of the patient's face. When the patient completes recording the video on the mobile computing device, the mobile computing device may transmit the video to the computer (as transmitted, a “video message”). The computer may store the video message, as well as all or some previous video messages, on a database which may further include anonymized data of other users. The computer includes software, which may implement some form of artificial intelligence, which can analyze the instant video message(s). Analysis of the video message(s) may include a categorization of the patient's facial expression(s) during the recording; said categorization may be in a numerical form (e.g., 1 corresponds to an angry facial expression). The patient's facial expression(s) can be compared with the textual and/or audio content of the same individual recording to “train” the system, though other means of training the system are not beyond the scope of the present disclosure. “Training the system may result in the system associating certain facial expressions with certain textual or audio messages. For example, the system may associate happy facial expressions with words such as “excited” or “wonderful.” This training, which may be combined with other data, such as whether the patient has a history of mental health diagnoses, can generate a probability of correlation between the expressive element and content of a given message. These probabilities (or “predictions”) can be used to generate CBT or other messages (e.g., alerts to emergency service providers) at the time the patient is making the video recording or shortly thereafter.

After the patient sends their video message to the computer, the computer may transmit a message to the mobile computing device. The message may mention an inconsistency between the patient's facial expression(s) or expressive elements and the content in their message and what the computer would have expected (or predicted) their facial expression to be, based on previous video messages and generalizations drawn from anonymized data. For example, if a patient sent video messages with content indicating a positive attitude though facial expression(s) or other expressive elements indicate a non-positive attitude, the system might predict that the patient does not in fact have a positive attitude. A mismatch between the system's expectation or prediction could inform the CBT reply to make said reply more accurate. For example, the reply could acknowledge the mismatch and refer to the discrepancy in expressive elements and content.

Embodiments of a computerized CBT system according to the present disclosure are not limited to a specific mode of communication. Such a system can support various communication platforms, such as a proprietary web app, WhatsApp, SMS (Simple Message Service), RCS (Rich Communication Services), iMessages, Signal, FaceTime or other text, voice, and/or video modalities. Thus, a computerized CBT system according to aspects of the present disclosure may allow partners to choose their preferred communication method. Speech-to-text, text-to-speech, and text-to-video technologies enable consistent and seamless interaction across different platforms and enhance accessibility by catering to diverse user preferences and needs. The disclosed computerized CBT system delivers a cohesive user experience regardless of the communication channel used.

A multi-agent approach is a key aspect of the present disclosure. In the computerized CBT interaction, each reply is computed not in a single step but through a complex interplay of multiple agents. These agents distribute intermediate “cognitive” steps across multiple specialized requests to generate a supportive reply. Each agent is specialized in handling specific aspects of the reply-generation task, contributing to a more accurate and efficient overall response. The system can adapt to different support scenarios by reconfiguring the agents and their interactions. By distributing tasks among multiple agents, the system enhances resilience and fault tolerance, reducing the impact of any single point of failure. Specialized agents improve the likelihood that each aspect of the support algorithm is addressed with the highest level of expertise, improving the overall accuracy and effectiveness.

Key agents include a summarizer, an inner voice, a curated content injector, a composer, and a supervisor.

The summarizer is configured to generate a diagnostic narrative from a series of messages and replies. Thus, the summarizer forms a summary of the case or conversation between the computerized CBT system and the partner. The summarizer also forms a partner profile, a comprehensive vector of relevant characteristics across various categories or dimensions of persona, demographics, goals, and limitations. Additionally, the summarizer detects and/or predicts missing information and generates anamnesis (guided recall) questions that can be fed to the composer. Overall, the summarizer provides a long-term memory representation of the computerized CBT system's interaction with the partner. As part of the long-term memory representation, the summarizer compresses the information from the messages and replies into a compact vector that can be fed to the composer. The compressed information enables maintenance of continuity in the conversation by keeping track of the partner's history, attributes, progress. The summarizer's representation of the interaction also enables provision of insight into the interaction. The summarizer operates in parallel to the other agents, so that its algorithm does not drive latency in the conversation.

The inner voice is configured to represent the cognitive process of an expert interlocutor. As such, the inner voice combines all available partner information (including the summarizer's representation of such information) with relevant professional knowledge to provide an expert assessment of the interaction and the partner's situation. Based on the expert assessment, the inner voice proposes relevant questions and/or suggestions that could be posed to the partner. The inner voice thereby plans a further course of action in the conversation. The inner voice operates independently of the summarizer, composer, and supervisor, working in parallel rather than sequentially. Once the inner voice formulates a new assessment, the assessment is stored in a history of the interaction for access and use by the composer. The inner voice, by operating in parallel to the other agents to plan the course of the conversation, enhances response speed from the partner's perspective by preparing assessments ahead of time. Unlike a human conversation, the system is fully capable of both receiving a message and planning a response in parallel. Thus, the inner voice enables enhanced or superior active listening.

The curated content injector (“CCI”) is an agent that responds to the expert assessment produced by the inner voice. The CCI provides pre-curated content elements such as: relaxation audios; in-depth motivational or information-seeking questions; conversational interventions; educational content; and/or instructions for responding to crises (e.g., in a business context, cash receipts less than cash expenses; in a psychotherapeutic context, suicidal ideation). The curated content can include, e.g., audio, image, and text elements in any combination; videos and interactive elements. The curated content may further include graphical (e.g., Cartesian coordinates of facial points) or image-based representations of human facial emotions. The CCI is intended to ensure that the partner receives pre-authored, well-targeted content exactly as intended by the authors. The CCI is further intended to provide a means to ensure that the content produced by the partner (or patient), in the forms of text or audio, correlates or does not correlate with the text, audio, or visual inputs provided by the partner (e.g., that the meaning of the text is statistically likely given the particular word combinations used, the time delay in producing those word combinations, and so on). The CCI matches content to the partner's situation based on a numerical matching (e.g., cosine distance) between a vector embedding of the inner voice's expert assessment and vector embeddings of an assessment by an LLM when to use this content—not of the content itself. Thus, the CCI provides a dynamic, automatic selection of conversational interventions and content, unprecedented in its capabilities. For example, the CCI may select the top five content elements that best fit (cosine distance match) the embedding of the current assessment of the partner's situation. The CCI then may provide these selected contents to the composer for potential inclusion in the response. In some embodiments, the composer may be obliged to include the curated content. In other embodiments, it may be optional for the composer to include the curated content.

The composer formulates drafts for a reply to the partner's message, based on all available information about the partner including the message itself, the inner voice's assessment and the summarizer's representation of the interaction with the partner. Thus, the composer utilizes information partially prepared by other agents. The composer tailors each reply to the specific needs and context of the partner. The composer maintains consistency in the conversation by harmonizing data from the other agents.

The composer does not send replies directly to the partner; instead, the supervisor reviews every reply and occasionally provides feedback to the composer. The supervisor generates feedback based on a set of relevant professional knowledge, which may be the same professional knowledge that is used by the inner voice. Checking replies against professional knowledge can help to make replies appropriate within the context of the conversation. Thus, the supervisor can protect the computerized CBT system against prompt injections and various malicious user requests. The supervisor is responsible for system boundary maintenance by ensuring that the overall system remains within the defined scope of the system's assigned purpose. The supervisor enhances the quality and safety of the system's replies, maintains consistent standards of expertise, and safeguards against potential misuse or harmful responses.

In response to feedback from the supervisor, the composer may produce revised draft replies.

Other features and aspects of the present teachings will become apparent from the following detailed description, taken in conjunction with the accompanying drawings, which illustrate by way of example the features in accordance with embodiments of the present teachings. The summary is not intended to limit the scope of the present teachings.

### BRIEF DESCRIPTION OF THE DRAWINGS

The present teachings are described more fully hereafter with reference to the accompanying drawings, which depict example embodiments. The following description illustrates the present teachings by way of example, not by way of limitation of the principles of the present teachings.

FIG. 1 depicts an overall interaction of a computerized CBT system 101 with a partner, consistent with selected aspects of the disclosure.

FIG. 2 depicts a high-level interaction 1001 of one embodiment of a computerized CBT system 101 comprised of a supervisor 110, summarizer 102, inner voice 104, composer 108, and curated content injector 106.

FIG. 3 depicts an overall interaction 300 of a computerized CBT system 101 with a partner 10, consistent with selected aspects of the disclosure.

FIG. 4 depicts an embodiment of the computerized CBT system 101 shown in FIG. 3.

FIG. 5 depicts inputs to a prompt 500 for an inner voice 104 of the computerized CBT system 101.

FIG. 6 depicts inputs to a prompt 600 for a composer 108 of the computerized CBT system 101.

FIG. 7 depicts inputs to a prompt 700 for a supervisor 110 of the computerized CBT system 101.

It should be understood that throughout the drawings corresponding reference numerals indicate like or corresponding parts and features.

### DETAILED DESCRIPTION

For purposes of explanation and not limitation, specific details are set forth such as particular structures, architectures, interfaces, techniques, etc. in order to provide a thorough understanding. In other instances, detailed descriptions of well-known devices and/or methods are omitted so as not to obscure the description with unnecessary detail.

CBT and other psychotherapeutic interventions can have profound effects on the patient's brain, and particularly on the limbic system (which is crucial for emotional regulation). CBT and other psychotherapeutic interventions can impact a patient in the following ways:

Emotion Regulation: The limbic system includes structures or components, such as the amygdala, hippocampus, and parts of the thalamus and hypothalamus, which are central to managing emotions. Psychotherapy can modify the responses of these limbic system structures to stress and emotional stimuli.

For example, psychotherapy may reduce the hypersensitivity of the amygdala to perceived (external) threats, thereby reducing feelings or manifestations of anxiety and depression in the patient.

Stress Response: Psychotherapy may alter how the limbic system reacts to stress. By changing thought patterns and emotional responses of a patient to stress, psychotherapy may reduce the activation of the hypothalamic-pituitary-adrenal (“HPA”) axis, which is often overactive in patients with chronic stress.

For example, psychotherapy may reduce excessive activations of the HPA axis, thereby decreasing cortisol levels in the patient and accordingly reducing the overall stress burden on the body.

Neuroplasticity: Through the process of neuroplasticity, psychotherapy may encourage the formation of new neural connections within the limbic system. These new neural connections change the manner in which the patient processes and expresses emotions in response to external stimuli.

For example, psychotherapy may increase connectivity between the prefrontal cortex and the limbic system, thereby enhancing the patient's ability to apply rational thought to control or modulate emotional reactions, resulting in an improvement in the patient's overall emotional stability.

Memory Processing: Psychotherapy may modify the manner in which a patient forms and processes memories. The hippocampus, a component of the limbic system, plays a significant role in forming new memories and processing emotional context. Techniques introduced to a patient through psychotherapy may allow a patient to better control reactions and expressions of already-present memories and re-shape those memories through integrating the emotional and factual aspects of the memories more effectively, thereby reducing the emotional intensity of those memories.

For example, psychotherapy may allow a patient to implement trauma-focused memory processing, thereby reshaping traumatic memories the patient has and allowing the patient to respond to those memories in a less intense manner.

Behavioral Changes: Psychotherapy may introduce changes in the limbic system which manifest in the patient's behavior (e.g., changes in behavior of the patient in response to inputs which are interpreted or processed through components of the limbic system). Changes to the emotional landscape of a patient's brain through psychotherapy may result in the patient finding it easier to engage in behaviors which were previously difficult to engage in.

For example, psychotherapy may allow a patient to remain calm in situations which (prior to treatment) would traditionally have caused panic in the patient. As another example, patients may also be able to alter habitual responses (e.g., drinking alcohol) to emotional triggers (e.g., stress).

Traditional psychotherapeutic interventions, including CBT, are typically limited by virtue of the provider-patient model where a patient must make an appointment with a doctor (or other professional) and the patient must further remember and accurately relay symptoms at this later date. Typically, this issue may be overcome by a patient maintaining a diary (or other form of contemporaneous recording) where symptoms and other aspects of the patient are stored. However, these types of solutions do not account for the patient themselves being an unreliable narrator, an issue exacerbated where the patient is suffering from a mental disease (e.g., depression). Accordingly, it is beneficial to offer the present invention which provides for an accounting of the emotional flexibility and expression of a patient and which may serve as a counterbalance to the unreliable narrator.

Generally, a patient will submit a short video, written message, audio recording, or some combination thereof on a regular basis (e.g., once per day). The present invention describes a system for analyzing the emotions or expressive elements presented by the patient and comparing these to the statements or content made by the patient at the same time, identifying incongruities, and using this data to implement a method of early detection for improvement or decline in the patient's condition. The system may further recommend a change in dosage or prescription type in accordance with the collected data.

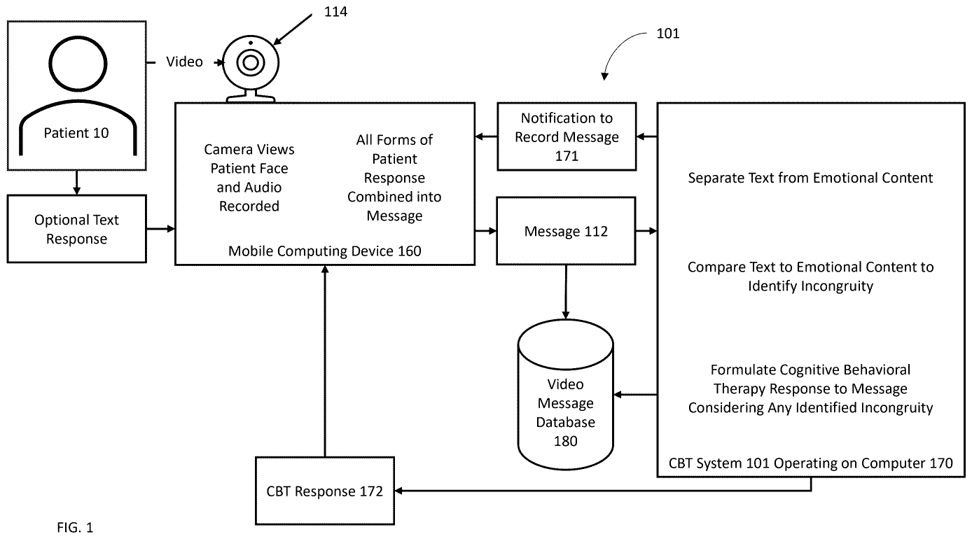

FIG. 1 depicts an exemplary embodiment of a system 101 according to the present disclosure. As seen in FIG. 1, a partner or patient 10 may interact with a camera 114. The camera 114 may be physically coupled with the mobile computing device 160 or may be electronically coupled or otherwise in communication. The camera 114 should be capable of capturing at least a portion of a partner's 10 face. The camera 114 may be capable of recording audio, but peripheral audio recording devices (e.g., an internal microphone of the mobile computing device 160) are within the scope of the present disclosure.

In an example of the operation of the interaction depicted in FIG. 1, a patient 10 will record a message 112. The message may be in response to a notification 171 from the computer 170 transmitted to the mobile computing device 160; alternatively, the message 112 may be recorded at any time spontaneously. The message 112 includes an image or video (with an audio component) recorded by camera 114 and a text response 113 which may consist of transcribed audio or typed text. The mobile computing device 111 equipped with a camera 114 receives the inputs from the partner 10, combining the elements into message 112 which is transmitted to the system 101. The system 101 may be further configured to provide a notification 115 to the partner 10 via mobile computing device 111 to prompt the partner 10 to record a message at a predetermined interval or in response to an identified incongruity in the prior message 112.

When the partner 10 completes the video recording, the mobile computing device 160 may transmit the video recording 112, as well as any audio and/or textual messages 113 to the computer 170. The entirety of the transmitted message, which may include audio and/or textual messages in addition to the video recording, is described as a video message 112. The video message 112 is transmitted from the mobile computing device 160 to the computer 170. The mobile computing device 160 may transmit the video message 112 to the computer 170 over a telecom network or any other known means of transmitting data.

The video message 112 is received by the computer 170. The computer 170 may be in communication with a video message database 180. Said video message database may be a database local to the computer 170 or may be cloud based or peripheral. The video message database 180 is preferably capable of storing a plurality of video messages 112. The video message database 180 is in communication with a computer 170. Said computer 170 may include software capable of retrieving prior video messages 112 from the video message database 180.

The computer 170 may transmit notifications 171 to the mobile computing device 160. The notification(s) 171 may be transmitted periodically (e.g., every day at 5 p.m.) or may be transmitted at random times or may be transmitted in response to an outside action (e.g., a physician or other person/program prompts the computer to send a notification 171). The notification 171 may include a message reminding the partner 10 to create a video recording.

The operation of the system involves analyzing both emotional and textual data from video and/or audio recordings. First, a traditional neural network detects emotions in the video or audio content and assigns timestamps to these emotions. Simultaneously, a large language model analyzes the spoken or textual content (which is also assigned timestamps) to capture the nuances of what is being communicated by the partner 10. By aligning these two timestamped data sets—emotional expression and textual content—any mismatches between what is ‘said’ by the partner 10 and how the partner 10 expresses what is ‘said’ become particularly insightful. For example, if the partner 10 says “haha, it doesn't bother me at all” while appearing visibly sad, this discrepancy or mismatch provides valuable information. The highlighted emotional incongruence between spoken words and facial expressions of the partner 10 provides an important indicator of emotional states that are not overtly acknowledged and allows the system to offer deeper insights into the partner's true feelings.

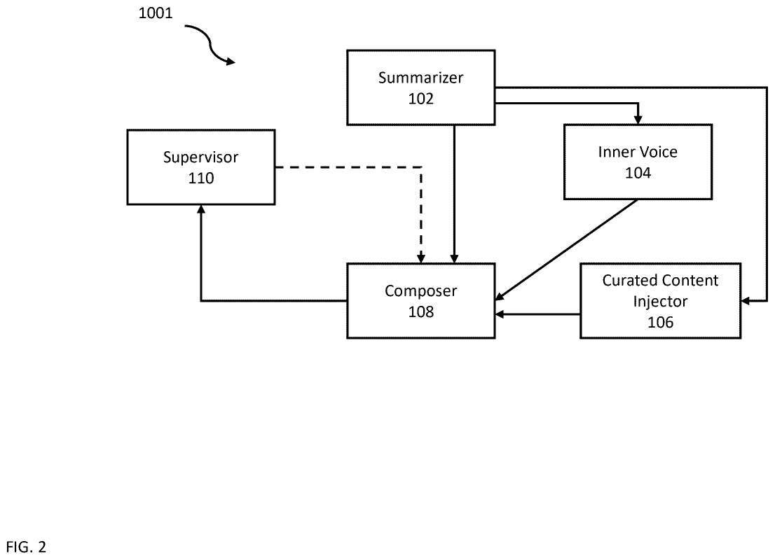

FIG. 2 depicts a high-level interaction 1001 of one embodiment of a computerized CBT system 101 comprised of a supervisor 110, summarizer 102, inner voice 104, composer 108, and curated content injector 106. In the depicted embodiment, lines of communication are shown. For example, the supervisor 110 may receive information, such as a draft reply 134, from the composer 108. In some instances, the supervisor may transmit supervisor feedback 138 to the composer 108. As shown, the summarizer 102, inner voice 104, curated content injector 106, and supervisor 110 may all be in communication with the composer 108 in a computerized CBT system 101 according to the present teachings.

In operation of the computerized CBT therapy system 101, the summarizer 102 receives the message 112 from the partner 10, which may occur through a messaging application 50. The message 112 includes one or more of text 114, sound 116, and/or video/image data 118. The summarizer 102 encodes the audio and/or video data as alt text and compiles the alt text with the message text 114 to form a full text 119. The summarizer sends the complete text 119 to the supervisor 110. The summarizer includes another encoder neural network 102.1 that is configured to compile the message 112 (optionally, in combination with sensed environmental factors 120) with one or more previous messages to produce an interaction (therapy) summary 122, which is a long-term memory representation of the interaction or conversation that the computerized CBT therapy system 101 has with the partner 10. The summarizer may also include a generative neural network 102.2 that may be configured to produce a partner (patient) profile 123 based on the interaction summary 122 using weights that are encoded with professional (therapeutic) knowledge 128. The summarizer 102 also may include a generative neural network 102.3 that is configured to identify gaps or missing information in the partner summary 122 and may be further configured to generate information-seeking or anamnesis questions 124 based on the partner summary 122. The summarizer 102 may be implemented, for example, as an encoder network. The summarizer 102 also may be implemented as a portion of a long, short term memory (LSTM) neural network. The summarizer 102 stores the partner summary 122 in a message history database 125 and also feeds the partner summary 122 to the inner voice 104.

The inner voice 104 is configured to generate an assessment of treatment factors 126 (“assessment”) including the partner and the interaction with the partner, based at least on the partner (patient) summary 122 and a set of professional (therapeutic) knowledge 128. The inner voice 104 may be configured, for example, as an encoder or as a transformer network that takes at least the partner summary 122 as a prompt. The set of professional knowledge 128 may be input to the inner voice 104 as a complex (many token, e.g., thousands of tokens) prompt, and/or may be encoded in the weights of the inner voice 104 in case the inner voice 104 is implemented as a large language model (LLM) or other type of neural network. The assessment 126 may be in the form of a multi-dimensional vector that diagnoses or describes the partner and the interaction across dimensions such as persona, demographics, goals, and limitations. The inner voice 104 feeds the assessment 126 to the composer 108, and also feeds the assessment 126 to the curated content injector 106.

The curated content injector 106 may match the assessment 126 to one or more items of curated content such as partner education 130 and/or risk response information 132, in order to identify any curated content that should be imparted to the composer 108. For example, the curated content injector 106 may vectorize the assessment 126 in a semantic space and then perform vector matching (e.g., cosine distance) between the vectorized assessment and respective semantic space vectors of the curated content.

The composer 108 is configured to receive at least the message 112, the partner summary 122, and the assessment 126, as well as (optionally) curated content 130, 132. The composer 108 may be implemented as a generative adversarial neural network (“GAN”) (e.g., using transformer architecture) that takes a compilation of the message 112, the partner summary 122, and the assessment 126 as a prompt, and may take the curated content 130, 132 either as an overriding prompt or as an addition to the prompt including the other content. The composer weights may be trained on a set of situational data, questions, and suggestions. The composer 108 is configured to deliver one or more draft replies 134 to the supervisor 110.

The supervisor 110 is configured to receive the draft replies 134 from the composer 108. The supervisor 110 may be implemented as a GAN that takes only the set of professional knowledge 128 and the current message 112 as inputs, produces a set of model replies, and uses a vector distance algorithm that compares each draft reply to each of the set of model replies. In case the supervisor 110 finds no close match, then the supervisor 110 may provide feedback 138 to the composer 108, thus prompting a revised set of draft replies.

At each iteration of message 112 and reply 113, the computerized CBT therapy system 101 stores these communications in the message history 125. The computerized CBT therapy system 101 also stores a compilation of patient summaries 122 in a treatment history 140.

A prototype of the computerized CBT therapy system may operate on multiple instances of GPT-4 by OpenAI. Open-source models such as LLaMA 3 are equally suitable. The computerized CBT therapy system may be self-hosted. Using multiple instances of large language models (LLMs) that take separate customized prompts and/or are trained on custom data enables the computerized CBT therapy system 101 to produce high-quality responses. LLMs can provide powerful capabilities for processing and generating human-like text. Moving to open-source models may enhance scalability and provide greater control over the system. For example, using a self-hosted open-source model may allow for customization and fine-tuning to meet specific support needs. Additionally, self-hosting ensures higher security and better privacy for user data. As an alternative or supplement to fine-tuning with data, embodiments of the computerized CBT therapy system may utilize advanced prompt engineering (for example, based on an database of curated prompts) for effective responses.

In various applications, certain components of the computerized CBT therapy system 101 may serve distinct roles. For example, if the computerized CBT therapy system 101 is implemented in a psychotherapeutic role, then the partner summary 122 may be better described as a patient summary 122, while the assessment 126 may be better described as treatment factors 126. In such an application, the curated content may be better described as patient education 130 and risk response 132.

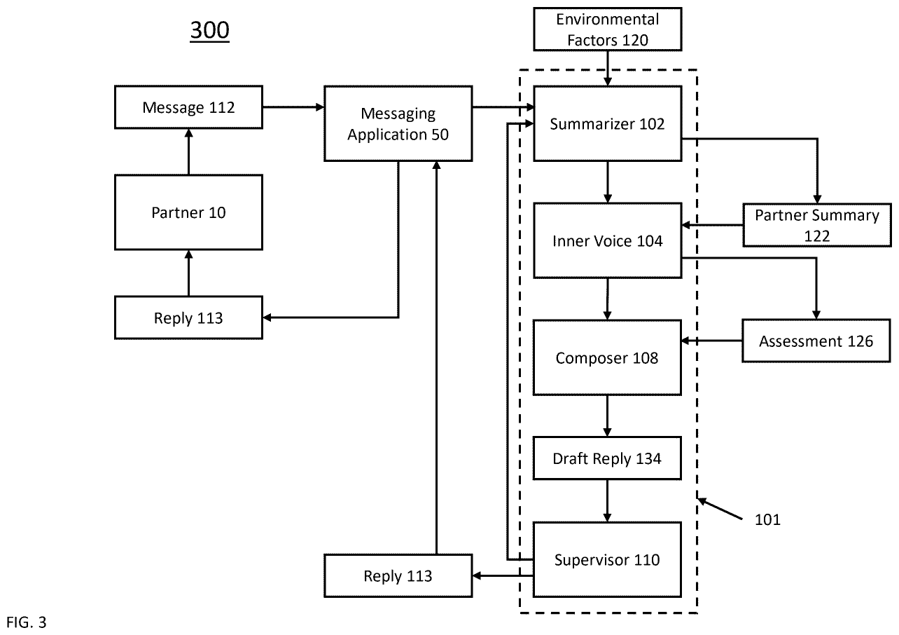

FIG. 3 depicts an overall interaction 300 of a computerized CBT system (operating on a computer 150) 101 with a partner (or “patient”) 10, consistent with selected aspects of the disclosure. The system 101 receives a message 112 from the partner 10 (e.g., a therapy patient), via a mobile computing device 50, and delivers a reply 113 to the partner 10. The system 101 produces the reply 113 based on one or more of the message 112, a partner (patient) summary 122 (not depicted in FIG. 1), and an image or video recorded by the camera 114 of the partner's face (or other visual cues) at the instant the message is delivered. The prompt 300 incorporates a therapeutic character 302, the therapeutic clinical narrative or assessment 126, a compilation of the last messages 306 (e.g., the six most recent messages), an echo of the last inner voice output 308, constraints and instructions 310, and a current time 312.

Options for the therapeutic character 302 include age, gender, race, education, and other aspects of a notional therapist's identity that are compiled into a framing portion of the prompt 300.

The therapeutic assessment 126 is an expert encoding or assessment of the message history as discussed above.

One purpose of the echo 308 is to maintain a continuity of context across multiple message and reply sequences.

The constraints and instructions 310 may include, for example, a constraint to acknowledge but not affirm negative messaging; a constraint to redirect attacks on the therapist/chat bot (where applicable); a constraint to ignore attacks on the therapist/chat bot (where applicable); an instruction to focus or perseverate on a given issue of concern to the partner/patient; an instruction to elicit additional detail from vague statements; etc.

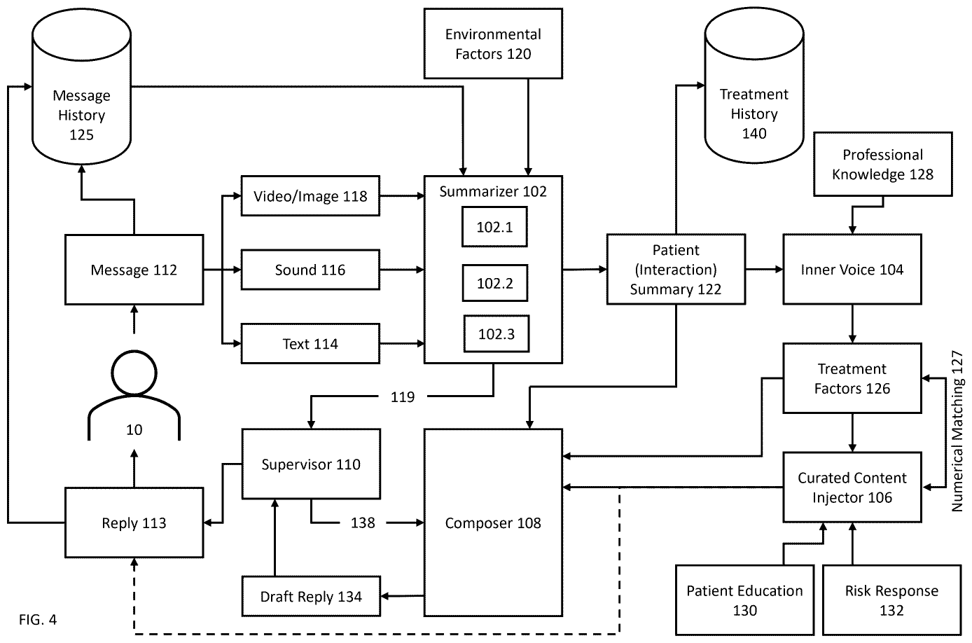

FIG. 4 depicts inputs to a prompt 400 for a composer 108 of the computerized CBT therapy system 101. The prompt 400 includes the therapeutic character 302, curated content 130, 132, therapeutic clinical narrative or assessment 126, inner voice output 404, missing information and anamnesis questions 124, constraints and instructions 310, time since last patient message 406, current time 312, supervisor feedback 138, and last patient messages 306.

As mentioned, the summarizer produces the anamnesis questions 124. The inner voice output 404 is produced by the inner voice 104 in response to the prompt 300.

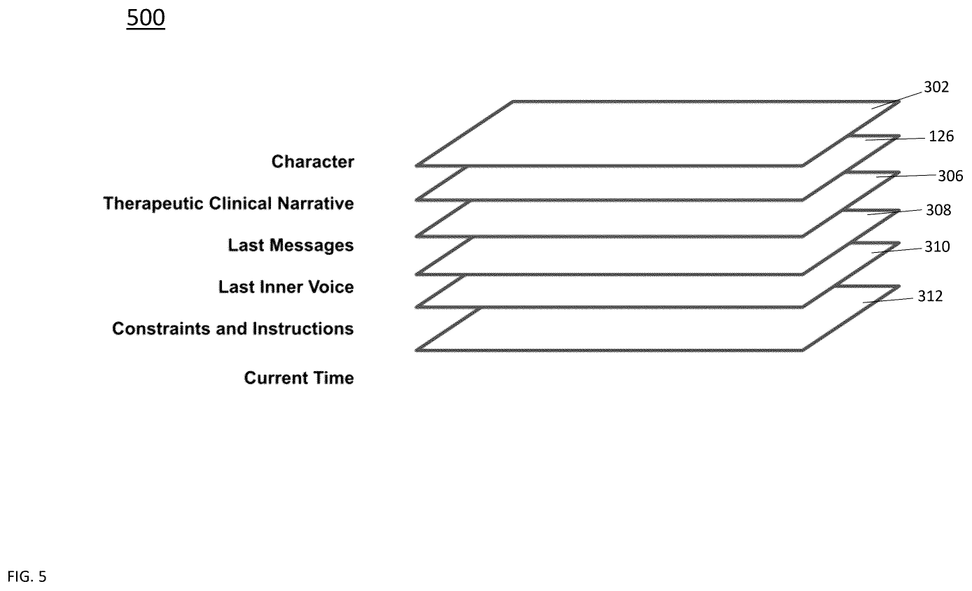

FIG. 5 depicts inputs to a prompt 500 for an inner voice 104 of the computerized CBT system 101. The prompt 600 incorporates a therapeutic character 302, the therapeutic clinical narrative or assessment 126, a compilation of the last messages 306 (e.g., the six most recent messages), an echo of the last inner voice output 308, constraints and instructions 310, supervisor character 502, time since last patient message 406, current time 312, and draft reply or replies 134, and a current time 312.

The supervisor character 502 is distinct from the therapeutic character 302 in at least one dimension of age, gender, race, education, or other identity factors. Advantageously, this gives the effect of multiple perspectives on the task at hand.

Options for the therapeutic character 302 include age, gender, race, education, and other aspects of a notional therapist's identity that are compiled into a framing portion of the prompt 600.

The therapeutic assessment 126 is an expert encoding or assessment of the message history as discussed above.

One purpose of the echo 308 is to maintain a continuity of context across multiple message and reply sequences.

The constraints and instructions 310 may include, for example, a constraint to acknowledge but not affirm negative messaging; a constraint to redirect attacks on the therapist/chat bot (where applicable); a constraint to ignore attacks on the therapist/chat bot (where applicable); an instruction to focus or perseverate on a given issue of concern to the patient/patient; an instruction to elicit additional detail from vague statements; etc.

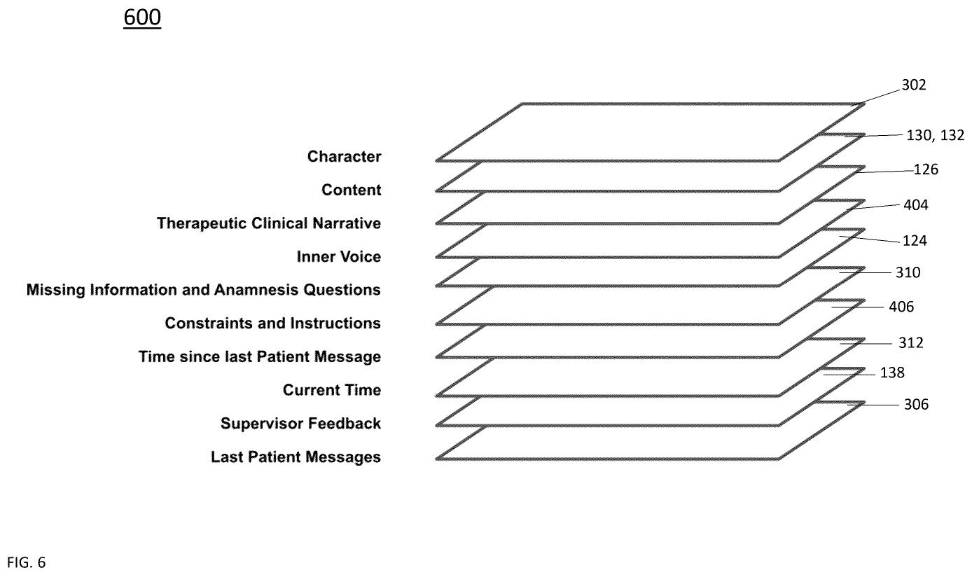

FIG. 6 depicts inputs to a prompt 600 for a composer 108 of the computerized CBT system 101. The prompt 600 includes the therapeutic character 302, curated content 130, 132, therapeutic clinical narrative or assessment 126, inner voice output 404, missing information and anamnesis questions 124, constraints and instructions 310, time since last patient message 406, current time 312, supervisor feedback 138, and last patient messages 306.

As mentioned, the summarizer produces the anamnesis questions 124. The inner voice output 404 is produced by the inner voice 104 in response to the prompt 600.

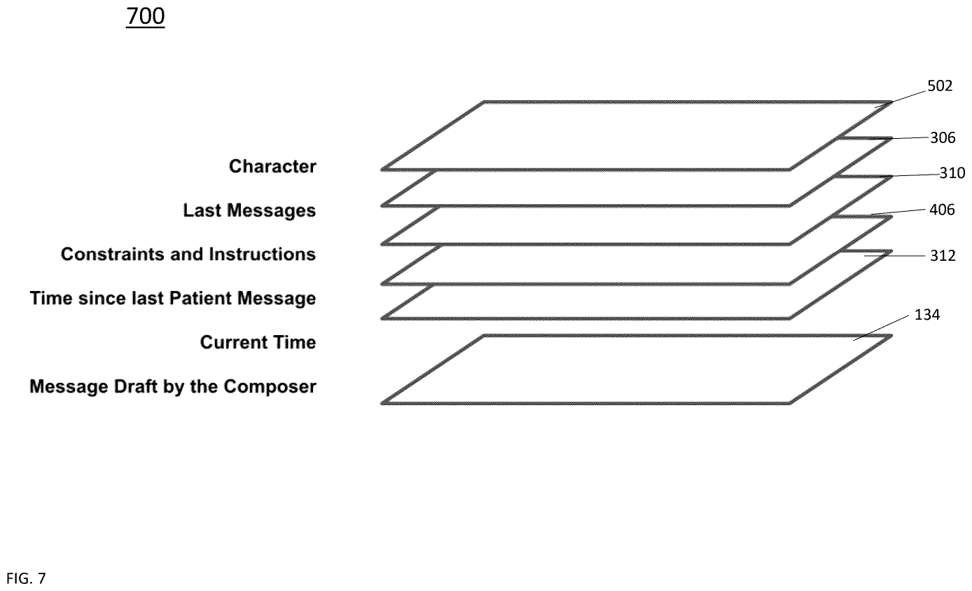

FIG. 7 depicts inputs to a prompt 700 for a supervisor 110 of the computerized CBT system 101. The prompt 700 includes supervisor character 502, last messages 306, constraints and instructions 310, time since last patient message 406, current time 312, and draft reply or replies 134.

The supervisor character 502 is distinct from the therapeutic character 302 in at least one dimension of age, gender, race, education, or other identity factors. Advantageously, this gives the effect of multiple perspectives on the task at hand.

The present teachings have been described in language more or less specific as to structural, mechanical, and functional features. It is to be understood, however, that the present teachings are not limited to the specific features shown and described, since the apparatus, system, and/or method herein disclosed comprises preferred forms of putting the present teachings into effect.

Generally, all terms used in the claims are to be interpreted according to their ordinary meaning in the technical field, unless explicitly defined otherwise herein. All references to a/an/the element, apparatus, component, means, step, etc. are to be interpreted openly as referring to at least one instance of the element, apparatus, component, means, step, etc., unless explicitly stated otherwise. The use of “first”, “second,” etc. for different features/components of the present disclosure are only intended to distinguish the features/components from other similar features/components and not to impart any order or hierarchy to the features/components, unless explicitly stated otherwise. The phrase “at least one of,” when used with a list of items, means that different combinations of one or more of the listed items may be used, and only one item in the list may be needed. For example, “at least one of: A, B, and C” includes any of the following combinations: A; B; C; A and B; A and C; B and C; and A and B and C.

Other than in the operating examples, or where otherwise indicated, all numbers expressing quantities of ingredients or reaction conditions used herein are to be understood as modified in all instances by the term “about”.

While the present teachings have been described above in terms of specific embodiments, it is to be understood that they are not limited to those disclosed embodiments. Many modifications and other embodiments will come to mind to those skilled in the art to which this pertains, and which are intended to be and are covered by both this disclosure and the appended claims. For example, in some instances, one or more features disclosed in connection with one embodiment can be used alone or in combination with one or more features of one or more other embodiments. It is intended that the scope of the present teachings should be determined by proper interpretation and construction of any claims and their legal equivalents, as understood by those of skill in the art relying upon the disclosure in this specification and the attached drawings.

What is claimed is:

1. A computerized cognitive behavioral therapy system comprising:
a computer in communication with a telecom network;
a patient message recorded by a patient, said patient message having a text portion and an emotional portion;
a mobile computing device for use by a patient, said mobile computing device receiving the patient message from the patient and transmitting said patient message to the computer over the telecom network;
software executing on the computer for separating the text portion from the emotional portion of said patient message and for comparing the message portions to identify incongruity and flag possible patient distress; and
software executing on the computer for formulating a cognitive behavioral therapy response based on both of the patient message text portion and any identified emotional incongruity.

2. The computerized cognitive behavioral therapy system of claim 1, wherein the emotional portion of the patient message is audio.

3. The computerized cognitive behavioral therapy system of claim 1, wherein the emotional portion of the patient message is video.

4. The computerized cognitive behavioral therapy system of claim 1, wherein the mobile computing device displays a periodic alert reminding a patient to submit a patient message.

5. The computerized cognitive behavioral therapy system of claim 1, wherein the patient provides a plurality of patient messages over a period of time.

6. The computerized cognitive behavioral therapy system of claim 5, wherein the computer is in communication with a database, and wherein said database stores the plurality of patient messages.

7. A computerized cognitive behavioral therapy system comprising:
a computer in communication with a telecom network;
a patient message recorded by a patient, said patient message having a text portion and an emotional portion;
a mobile computing device for use by a patient, said mobile computing device receiving the patient message from the patient and transmitting said patient message to the computer over the telecom network;
software executing on the computer for separating the text portion from the emotional portion of said patient message;
software executing on the computer for assigning a value to the emotional portion of the patient message based on the emotional flexibility present in said patient message;
software executing on the computer for assigning a value to the text portion of the patient message based on the emotional flexibility present in said patient message; and
software executing on the computer for formulating a cognitive behavioral therapy response based on the value assigned to the text portion of the patient message and the value assigned to the emotional portion of the patient message, said cognitive behavioral therapy response transmitted to the mobile computing device.

8. The computerized cognitive behavioral therapy system of claim 7, further comprising a database in communication with the computer for storing one or more patient messages.

9. The computerized cognitive behavioral therapy system of claim 8, further comprising software executing on the computer for identifying incongruity between the value assigned to the text portion of the patient message and the value assigned to the emotional portion of the patient message.

10. The computerized cognitive behavioral therapy system of claim 9, wherein the software can compare the value assigned to the text portion of the patient message and the value assigned to the emotional portion of the patient message with a plurality of values assigned from patient messages stored in the database.

11. A computerized cognitive behavioral therapy system comprising:
a computer in communication with a database containing a plurality of cognitive behavioral therapy messages;
a mobile computing device for use by a patient, said mobile computing device receiving the patient message from the patient and transmitting said patient message to the computer over the telecom network;
software executing on the computer for separating the text portion from the emotional portion of said patient message;
software executing on the computer for assigning a value to the emotional portion of the patient message based on the emotional flexibility present in said patient message;
software executing on the computer for assigning a value to the text portion of the patient message based on the emotional flexibility present in said patient message;
software executing on the computer for obtaining a cognitive behavioral therapy response from the database based on the value assigned to the text portion of the patient message and the value assigned to the emotional portion of the patient message, said cognitive behavioral therapy response transmitted to the mobile computing device.
